(19) United States
(12) Patent Application Publication
(10) Pub. No.: US 2004/0228799 A1
(43) Pub. Date: Nov. 18, 2004

(54) Dysphagia diagnostic kit and materials

(76) Inventor: Cynthia Armstrong, Longview, TX (US)

Correspondence Address: GEORGE S. GRAY, P.O. BOX 270190, CORPUS CHRISTI, TX, 78427-0190, US

(21) Appl. No.: 10/777,699

(22) Filed: Feb. 12, 2004

Related U.S. Application Data
(63) Continuation-in-part of application No. 09/166,554, filed on Oct. 5, 1998, now abandoned.
(60) Provisional application No. 60/061,708, filed on Oct. 6, 1997.

(51) Int. Cl. A61K 49/04; A61K 47/00; A61K 33/04

(52) U.S. Cl. 424/9.41; 424/709; 424/439

(57) ABSTRACT

A single use, dysphagia kit is provided for conducting a modified barium swallow, that provides a container of liquid barium sulfate solution of a thin liquid consistency, a container of liquid barium sulfate solution of a nectar consistency, a container of liquid barium sulfate solution of a honey consistency, a container of barium sulfate solution of a semi-solid consistency; a solid food substance containing barium sulfate, a plurality of spoons, a plurality of drinking straws, and a package for holding the ingredients. The solid food substance includes a cookie with barium sulfate icing, a sandwich cookie with barium sulfate filling, or a cookie with barium sulfate included as an ingredient during baking.

### REFERENCE TO PRIOR APPLICATION

This application claims the benefit of U.S. Provisional Application No. 60/061,078, filed Oct. 6, 1997. This application is a continuation-in-part application and also claims the benefit of the parent, i.e. U.S. application Ser. No. 09/166,554, filed Oct. 5, 1998.

### BACKGROUND OF INVENTION

This invention relates, in general, to a kit for clinical use in performing a modified barium swallow. What sets this invention apart from the products heretofore used is that the invention, in its preferred embodiment, is a single use kit that contains the five diagnostic mediums that are used in performing modified barium swallow, dysphagiagrams and video fluoroscopic swallow studies.

Each of the separate elements in the pack are known to the art: however, there are no known diagnostic kits containing the five commonly used barium product consistencies that are necessary for performing a complete dysphagia diagnostic study. This kit fills a need that exists in this diagnostic area. One of the important elements of clinical diagnosis is that the data must be objective, quantitative, and reproducible. Further, it is highly desired that items needed for diagnosis be easily obtainable, stable, and available when and where needed. The present invention fills those needs.

Performance of the modified barium swallow entails having a patient imbibe different consistencies of barium sulfate while undergoing videofluorography or X-ray analysis. The present method involves having the practitioner mix several containers of barium sulfate into four consistencies, and spreading a barium paste on a solid food substance, usually a cookie. In the literature, the consistencies are variously described as thin, thick, honey consistency, nectar consistency, and pudding consistency or paste.

Patients, who have difficulty swallowing, or those who are aspirating certain food substances are generally the candidates for differential diagnosis using the modified barium swallow. Such difficulties are seen in stroke patients, head injuries, cancer, and other diseases and injuries that effect the muscles and nerves of the neck. These patients are asked to swallow the different consistencies of barium sulfate while undergoing radiographic analysis so that the resulting radiograms show which phase of swallowing, and to what degree (as seen by aspiration of the test material), is effected by the disease or injury.

The procedure, as commonly performed in most clinical situations, allows for a fair amount of subjectivity, thereby lowering the reproducibility of the results. Further, a practitioner must have available both pudding and a cookie in order to make all of the required consistencies necessary for a complete differential diagnosis of swallowing pathologies. Further, most clinicians find it necessary to have a flavoring agent (such as fruit juice) on hand because barium sulfate is not palatable. The present invention solves this problem by including four different liquid consistencies preferably mixed with a flavoring agent, and one barium sulfate impregnated solid food substance in the kit, so that everything is right at hand and premixed to a determined consistency. The present invention provides the practitioner with a readily available set of premixed variable consistency barium sulfate. Each kit, of the same series, would have the same set of consistencies of barium sulfate; therefore, reproducibility of test results is enhanced.

### SUMMARY OF THE INVENTION

The present invention provides a diagnostic kit containing multiple containers (all containers approximately 8 ounces, but could be any other appropriate size) of barium sulfate mixtures wherein each container contains a barium sulfate solution of a different consistency. In addition, the preferred embodiment includes a flavoring agent to make each of the barium sulfate mixtures more palpatable. In the preferred embodiment, said diagnostic kit consists of four containers wherein on container consists of “thin liquid” consistency barium sulfate mixture (approximately 30% w/v), one container “nectar” consistency barium sulfate mixture (approximately 150% w/v), one container “honey” consistency barium sulfate mixture (approximately 250% w/v), one container of a “pudding thick” barium sulfate paste (or esophageal cream), one cookie (or similar solid food substance) that is either impregnated with a barium sulfate, covered with barium sulfate (usually in a paste form applied in a manner similar to “icing”) or has barium sulfate paste sandwiched between two food layers. In the preferred embodiment the kit would also contain plastic spoons and two drinking straws.

The barium sulfate impregnated solid food substance can be made from any commercially feasible recipe, including ingredients to mask the taste of the barium sulfate. Alternatively, the solid food substance can be made like a sandwich with a barium sulfate paste spread between two solid food layers, or barium sulfate paste folded into a pocket or layered on the top in the manner of “icing” on a cookie or cake. For use in the modified barium study, it is best to use a hard type cookie, although any food substance that chews into small hard particles would do. A cookie is chosen primarily because it is economically feasible to manufacture, and it is appealing to most patients. The cookie can be hermetically sealed in plastic to keep it sterile.

The barium sulfate paste, can be made from any paste like substance, but to mask the taste of the barium sulfate the present invention contemplates the use of a commercial pudding recipe, particularly a pudding that does not require refrigeration and that has a long shelf life.

The liquid barium consistencies are formed by conventional mixing methods, that include a palatable liquid, such as apple juice, Kool-Aid, lemonade, or any similar liquid, including plain water. Viscosity increasing thickeners are known. Again, flavored liquids are preferred in order to mask the barium sulfate taste.

The packaging can be constructed from any commercially feasible material that will provide the required degree of sterility, hardiness, and any other manufacturing factor that need be considered. Likewise, the individual containers can be of any commercially feasible material suitable for the clinical setting.

### BRIEF DESCRIPTION OF THE DRAWINGS

FIG. 1 is an oblique three dimensional view of the dysphagia pack and its contents.

FIG. 2 is a side elevation of one type of a sandwich type cookie containing a barium laced filling.

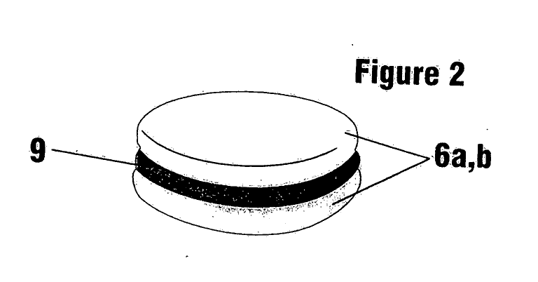

### DETAILED DESCRIPTION OF PREFERRED EMBODIMENTS

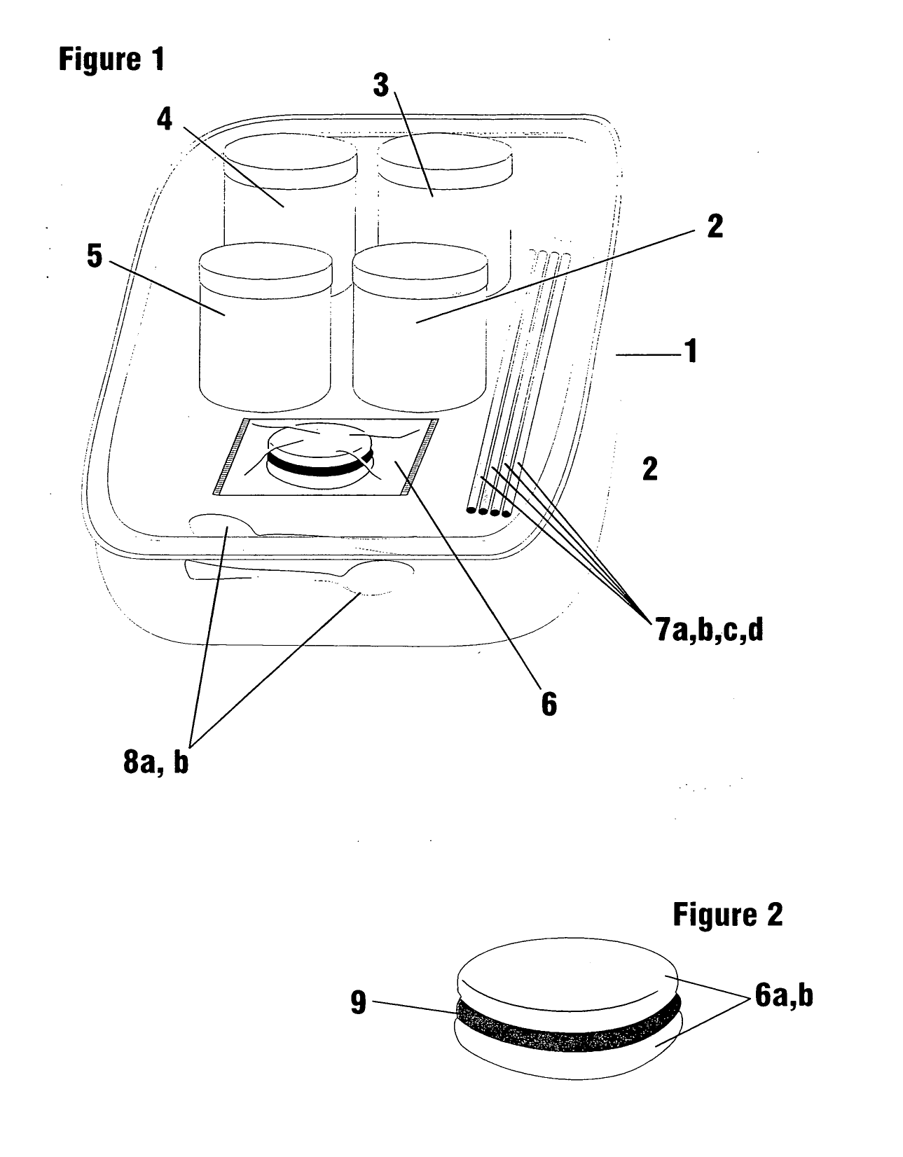

In accordance with FIG. 1, a dysphagia pack is constructed of a container (1) that can be made of plastic or any other material suitable for the clinical setting. The container should be constructed such that a top or cover can be sealed after the other elements are placed into the container. The container may also be constructed in a manner such that it may be sterilized, or otherwise made ready for clinical use. The contents of the dysphagia pack include four containers (2-5) holding barium sulfate wherein each container holds one of the viscosities of barium sulfate solution used in the modified barium swallow test. Each of the containers is matched for one of the prescribed steps used in the modified barium swallow. The containers need to be of a size appropriate for the diagnostic purpose. The containers can be jars, tube, or any other suitable conveyance. The purpose of the container is merely to provide the clinician with barium sulfate solutions for each of the necessary steps, so that the clinician can perform a modified barium swallow without the need to mix various solutions. The dysphagia pack also includes a cookie (6) that has been impregnated or coated with barium. The ideal dysphagia pack will also include four straws (7a-d) and two spoons (8a-b) for use by the patient.

The preferred embodiment of this dysphagia pack would be a package manufactured to conveniently hold four containers of barium sulfate, one cookie iced with barium sulfate paste, four plastic spoons, and two plastic drinking straws. Each of the four containers would hold different consistencies of barium sulfate. A container, of approximately 250 ml, “thin” consistency barium sulfate liquid in the range of 30% w/v barium sulfate, i.e. 20-40 w/v barium sulfate. A second container containing 250 ml of a “nectar” consistency barium sulfate solution. A third container of “honey” consistency containing 250 ml of a barium sulfate solution. A fourth container containing 125 ml of barium sulfate paste, evenly dispersed in a commercial pudding that does not need to be kept refrigerated. The dysphagia pack would also contain one cookie that was produced from a commercial recipe thereby approximately 20 ml of barium paste is sandwiched between two cookie layers. The barium paste “filling” may be mixed with sweeteners or other taste masking agents. Alternatively, a cookie may be baked, using any commercially feasible recipe, that contains ½ (2.5 ml) teaspoon barium sulfate thoroughly mixed in the ingredients prior to baking. The cookie should be sealed in a plastic wrapping to keep it clean and fresh.

I claim:

1. A dysphagia pack comprising:
A container of liquid barium sulfate solution of a thin liquid consistency;
A container of liquid barium sulfate solution of a nectar consistency;
A container of liquid barium sulfate solution of a honey consistency;
A container of barium sulfate solution of a semi-solid consistency;
A solid food substance containing a barium sulfate;
A plurality of spoons;
A plurality of drinking straws; and,
A package for holding the ingredients.

2. A dysphagia pack as described in claim 1, wherein:
The solid food substance is a cookie made from a conventional recipe that contains a barium sulfate solution as part of the recipe, such that said cookie is impregnated with radioactive dose of barium.

3. A dysphagia pack as described in claim 1, wherein:
The solid food substance is covered on one or more surface with a coating of a barium sulfate paste.

4. A dysphagia pack as described in claim 1, wherein:
The solid food substance is made in two layers with a barium sulfate paste interposed between the two layers.

5. A dysphagia pack as described in claim 1 wherein:
The solid food substance is two layers of cookie made from any conventional recipe whereby a layer of barium sulfate paste is placed between the two cookie layers.

6. A dysphagia pack as described in claim 1, wherein:
The barium sulfate of a semi-solid consistency is a commercial pudding mixture that contains a radioactive amount of barium sulfate.

7. A dysphagia pack as described in claim 1, wherein:
The barium sulfate solution of a semi-solid consistency is a commercial esophageal paste of approximately 60% w/w barium sulfate that has been flavored with food additives to make said paste palatable.

8. A dysphagia pack as described in claim 1, wherein:
The liquid used to dissolve the barium sulfate contains a flavored food additive substance that masks the taste of the barium sulfate in a manner that makes the mixture palatable.

9. A dysphagia pack as described in claim 1, wherein:
The thin liquid comprises a solution of a barium sulfate with a concentration of approximately 20% to 40% w/v barium sulfate.

10. A cookie for use in a modified barium study, comprising a cookie into which barium sulfate has been baked, in such a manner and in such quantities, such that the cookie crumbles into hard particles as it is being chewed.
